(19) United States
(12) Patent Application Publication
(10) Pub. No.: US 2024/0266059 A1
(43) Pub. Date: Aug. 8, 2024

(54) GENE-BASED DIGITAL TWIN SYSTEM THAT CAN PREDICT MEDICAL RISK

(71) Applicant: PREDICTIV CARE, INC., Mountain View, CA (US)

(72) Inventor: Sijung YUN, Mountain View, CA (US)

(21) Appl. No.: 18/298,462

(22) Filed: Apr. 11, 2023

Related U.S. Application Data
(63) Continuation of application No. PCT/US23/65394, filed on Apr. 5, 2023.

(30) Foreign Application Priority Data
Feb. 7, 2023 (KR) 10-2023-0016382

(51) Int. Cl. G16H 50/30; G16H 70/40

(52) U.S. Cl. CPC G16H 50/30; G16H 70/40

(57) ABSTRACT

The present disclosure relates to a gene-based digital twin system which is capable of predicting a medical risk about a disease possibility or the medical risk of the drug of a patient. To this end, provided is a gene-based digital twin system which is capable of predicting a medical risk, including a clinical information generating unit, including, (i-1) a DNA preprocessing unit which processes genetic sequencing data of a patient in a predetermined format, (i-2) a bioinformatics pipeline unit which sequentially outputs the preprocessed DNA bio information in a predetermined unit, (i-3) a predicted candidate group screening AI unit which screens a predicted candidate group for the disease and a predicted candidate group for the drug with respect to the DNA bio information output from the bioinformatics pipeline unit, (i-4) a dimensionality reduction AI unit which reduces a size of the preprocessed DNA bio information, and (i-5) a bioinformatics integration unit which receives and integrates the bio information trained by the predicted candidate group screening AI unit and the dimensionality reduction AI unit from the bioinformatics pipeline unit, (ii) a patient's digital twin file which is virtually generated based on information about a body of the patient, a predicted candidate group for the disease and a predicted candidate group for the drug received from the bioinformatics integration unit, (iii) a disease information table which receives and stores information about a disease from the outside, (iv) a drug information table which receives and stores information about a drug from the outside, and (v) a controller which predicts and outputs a medical risk of the patient based on the digital twin file, the disease information table, and the drug information table.

### TECHNICAL FIELD

The present disclosure relates to a system which is capable of predicting a disease possibility or a drug risk of a patient, and more particularly, to a gene-based digital twin system which is capable of predicting a medical risk.

### BACKGROUND ART

Generally, a patient used to cure the disease by directly taking the medicine prescribed by a doctor. Accordingly, even though a patient has a side effect to a specific medicine due to the physical constitution, disease, health condition, and age of the patient, the side effect may be confirmed only by taking the medicine. That is, the patient has to accept the anxiety and the risk of a medicine to be taken for the first time.

Further, even for the medicine which is widely generalized and used (for example, Tylenol, and Aspirin), the side effects which may occur with a low probability cannot be avoided. Patients who experience the rare side effects will inevitably damage their health by taking the medicine. Recently, in order to minimize the drug side effects, services are provided to check and guide concomitant prescription or concurrent medication in advance. However, these services had limitation to be provided based on only the chemical reaction between drugs by ignoring the characteristic of an individual patient.

Further, it is known that a specific drug does not respond well to a patient with a certain genetic variant. Despite such academic knowledge, the genetic test is not performed on all patients before administration, and the test is practically impossible.

In the meantime, in some cases, genetic causes or family history may cause a high possibility of specific diseases. For example, obesity genes may cause an adult disease, certain cancer disease, or glaucoma. In this case, even though a current health condition of the patient is good, consistence management and observation are requested.

### DISCLOSURE

### Technical Problem

The present disclosure has been made an effort to solve the above-described problems, and an object to be achieved by the present disclosure is to provide a gene-based digital twin system which previously predicts a medical risk by implementing a digital twin by coupling a gene and a physical characteristic.

Another object of the present disclosure is to provide a gene-based digital twin system which may predict a medical risk by applying a next generation sequencing (NGS) technique to a patient's gene, coupling individual physical characteristics to configure a genome of the patient in a digital form to previously predict the individual susceptible diseases and/or potential drug side effects.

Technical objects to be achieved in the present disclosure are not limited to the aforementioned technical objects, and another not-mentioned technical object will be clearly understood by those skilled in the art from the description below.

### Technical Solution

In order to achieve the above-described technical objects, provided is a gene-based digital twin system which is capable of predicting a medical risk including, a clinical information generating unit, including, (i-1) a DNA preprocessing unit which processes genetic sequencing data of a patient in a predetermined format; (i-2) a bioinformatics pipeline unit which sequentially outputs the preprocessed DNA bio information in a predetermined unit; (i-3) a predicted candidate group screening AI unit which screens a predicted candidate group for the disease and a predicted candidate group for the drug with respect to the DNA bio information output from the bioinformatics pipeline unit; (i-4) a dimensionality reduction AI unit which reduces a size of the preprocessed DNA bio information; and (i-5) a bioinformatics integration unit which receives and integrates the bio information trained by the predicted candidate group screening AI unit and the dimensionality reduction AI unit from the bioinformatics pipeline unit, (ii) a patient's digital twin file which is virtually generated based on information about a body of the patient, a predicted candidate group for the disease and a predicted candidate group for the drug received from the bioinformatics integration unit; (iii) a disease information table which receives and stores information about a disease from the outside; (iv) a drug information table which receives and stores information about a drug from the outside; and (v) a controller which predicts and outputs a medical risk of the patient based on the digital twin file, the disease information table, and the drug information table.

Further, the predicted candidate group screening AI unit screens the predicted candidate group for the disease and the predicted candidate group for the drug for a predetermined major gene variant among the DNA bio information.

Further, the predetermined major gene variant includes a list including at least one of a name of the gene, a variant type, a variant ID, a zygosity type, a chromosome number, a position, and a genotype.

Further, the predicted candidate group for the disease is screened by dividing the risk of the disease into a plurality of levels. The plurality of levels is divided into a first level of susceptible disease group having a highest disease onset possibility; a third level of susceptible disease group having a lowest disease onset possibility; and a second level of susceptible disease group having a possibility corresponding to an intermediate level between the first level of susceptible disease group and the third level of susceptible disease group.

Further, the predicted candidate group for the drug is screened by dividing the risk of the drug side effects into a plurality of levels.

The plurality of levels is divided into a first level of risk drug group having a highest drug side effect possibility; a third level of risk drug group having a lowest drug side effect possibility; and a second level of risk drug group having a possibility corresponding to an intermediate level between the first level of risk drug group and the third level of risk drug group.

Further, the predicted candidate group for the drug includes at least one of information about a toxicity, a dosage, an efficacy, and metabolism of the drug.

Further, the digital twin file further includes a 3D human body avatar model.

Further, information about the disease or the drug is displayed on the 3D human body avatar model.

### Advantageous Effects

According to an exemplary embodiment of the present disclosure, a patient may be notified in advance of susceptible diseases which are highly likely to develop by the digital twin of the patient. By doing this, an individual may seek to improve dietary life, regular exercise, and change in lifestyle according to their individual characteristics, and may respond early in the event of an onset through constant examination of the disease.

Further, the patient may be notified in advance of the possibility of the drug side effects through the digital twin of the patient. Specifically, even though it is a widely used drug, the possibility of ineffectiveness or side effects depending on the characteristics of the patient's genes may be blocked in advance.

Accordingly, the digital twin system may significantly reduce the medical risks in real time during the doctor's treatment or prescription.

A technical object to be achieved in the present disclosure is not limited to the aforementioned effects, and another not-mentioned effects will be obviously understood by those skilled in the art from the description below.

### DESCRIPTION OF DRAWINGS

The following drawings attached to the present specification illustrate the exemplary embodiment of the present disclosure and are provided for better understanding of the technical spirit of the present disclosure together with the detailed description of the disclosure to be described. Therefore, the present disclosure should not be construed as being limited only to the matters described in the drawings.

FIG. 1 is a schematic block diagram of a digital twin system according to an example embodiment of the present disclosure.

FIG. 2 is a diagram illustrating a main screen 500 of a digital twin system according to an example embodiment of the present disclosure.

FIG. 3 is a diagram illustrating a clinical note unit 600 displayed by a document generation button 545 of FIG. 2 according to an example embodiment.

FIG. 4 is a diagram illustrating a genetic variant list 700 displayed by a genetic information icon 590 of FIG. 2 according to an example embodiment.

FIG. 5 is a diagram illustrating a susceptible disease predicting unit 800 displayed by a susceptible disease icon 580 of FIG. 2 according to an example embodiment.

FIG. 6 is a diagram illustrating a detailed disease informatics unit 850 displayed by clicking one disease of FIG. 5 according to an example embodiment.

FIG. 7 is a diagram illustrating a drug risk predicting unit 900 displayed by a drug icon 585 of FIG. 2 according to an example embodiment.

FIG. 8 is a diagram illustrating a detailed drug informatics unit 1000 displayed by clicking one drug of FIG. 7 according to an example embodiment.

FIG. 9 is a diagram illustrating a scheduling window 1100 displayed by clicking a scheduling button 575 of FIG. 2 according to an example embodiment.

### BEST MODE

In the following detailed description, only certain exemplary embodiments of the present disclosure have been shown and described, simply by way of illustration. Description of the present disclosure is just an embodiment for structural and functional description so that the scope of the present disclosure is not interpreted to be limited by the embodiment described in the specification. That is, the embodiment may be modified in various forms so that it is understood that the scope of the present disclosure has equivalents which are capable of implementing the technical spirit. Further, it does not mean that the specific embodiment includes the object or effect proposed in the present disclosure or includes only the effect so that it is not understood that the scope of the present disclosure is limited thereby.

In the meantime, meanings of terms described in the present disclosure can be understood as follows.

The terms “first” or “second” are used to distinguish one component from the other component so that the scope should not be limited by these terms. For example, a first component may be referred to as a second component, and similarly, a second component may be referred to as a first component. It should be understood that, when it is described that an element is “connected” to another element, the element may be directly connected to the other element or connected to the other element through a third element. In contrast, it should be understood that, when it is described that an element is directly connected to another element, no element is present between the element and the other element. Other expressions which describe the relationship between components, that is, “between” and “directly between”, or “adjacent to” and “directly adjacent to” need to be interpreted by the same manner.

Unless the context apparently indicates otherwise, it should be understood that terms “include” or “have” indicate that a feature, a number, a step, an operation, a component, a part or the combination thereof described in the specification is present, but do not exclude a possibility of presence or addition of one or more other features, numbers, steps, operations, components, parts or combinations thereof, in advance.

Unless they are contrarily defined, all terms used herein including technological or scientific terms have the same meaning as those generally understood by a person with ordinary skill in the art. Terms which are defined in a generally used dictionary should be interpreted to have the same meaning as the meaning in the context of the related art but are not interpreted as an ideally or excessively formal meaning if it is not clearly defined in the present disclosure.

Hereinafter, preferred exemplary embodiments will be described in detail with reference to the accompanying drawings.

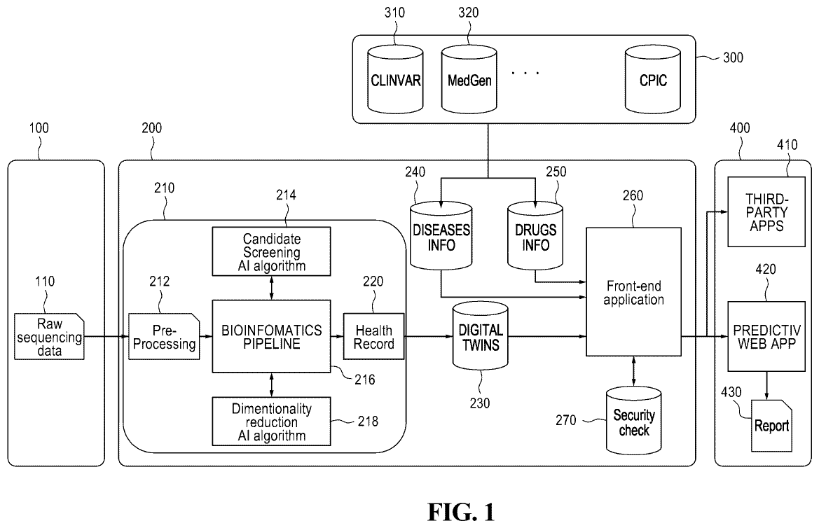

FIG. 1 is a schematic block diagram of a digital twin system according to an example embodiment of the present disclosure. As illustrated in FIG. 1, a DNA sequencing analysis unit 100 extracts a DNA from a fingernail of a specific patient to generate DNA sequencing data (raw sequencing data) 110.

The digital twin predicting unit 200 generates a digital twin file and provides a service for a medical risk. The digital twin predicting unit 200 may be a server computer connected to a network (for example, internet) and bi-directionally communicate with a public DB 300. The digital twin predicting unit 200 is a platform for a gene-based digital twin system.

A DNA preprocessing unit 212 preprocesses the DNA sequencing data 110 to a specific format to be computed in a bioinformatics pipeline unit 216. For example, the DNA preprocessing unit 212 applies a next generation sequencing (NGS) technique to an individual DNA and couples a physical characteristic of the individual to configure an individual genome as a digital form.

The bioinformatics pipeline unit 216 modulates the preprocessed DNA sequences in a predetermined unit to sequentially output the preprocessed DNA sequences. The predetermined unit of DNA sequences is transmitted to a predicted candidate group screening AI unit 214 and a dimensionality reduction AI unit 218, and completed data is transmitted to a bioinformatics integration unit 220.

The predicted candidate group screening AI unit 214 is an artificial intelligence model which screens a predicted candidate group for the disease and a predicted candidate group for a drug with respect to the DNA bio information transmitted from the bioinformatics pipeline unit 216. To this end, the predicted candidate group screening AI unit 214 performs machine learning on numerous medical information, clinical information, and genetic information through a neural network and a convolution neural network (CNN). To be more specific, the predicted candidate group screening AI unit 214 performs machine learning on 20000 pieces of genetic information, 22000 pieces of disease information for human and 750 types of drug information approved by the FDA. Further, the predicted candidate group screening AI unit 214 periodically learns latest medical information to enable the reasoning based on the latest information.

The dimensionality reduction AI unit 218 serves to reduce approximately 3 billion pieces of DNA information provided from the bioinformatics pipeline unit 216 to predetermined 20000 pieces of genetic information. The dimensionality reduction AI unit 218 enables the rapid prediction by avoiding unnecessary computations to save a computing resource.

The bioinformatics integration unit 220 receives and integrates bio information trained by the predicted candidate group screening AI unit 214 and the dimensionality reduction AI unit 218, from the bioinformatics pipeline unit 216. The digital twin 230 for the patient is generated by the integration described above.

A clinical information generating unit 210 includes, aforementioned, the DNA preprocessing unit 212, the predicted candidate group screening AI unit 214, the bioinformatics pipeline unit 216, the dimensionality reduction AI unit 218, and the bioinformatics integration unit 220. The clinical information generating unit 210 generates a digital twin 230.

The digital twin is a model file in which genetic information and physical characteristic information of the patient are coupled, and the digital twin matches the corresponding patient one to one. The digital twin allows finding a susceptible disease of the patient in advance and identifying a drug side effect in advance. Accordingly, the digital twin may also be called a patient bio information file, a digital pseudo-body, or a virtual patient model.

The public database 300 is a medical database service provided from external institutions. For example, the public data may be CLINVAR 310 which is an open archive providing interpretation of human genetic variation and disease from National Center for Biological Information (NCBI) in the United States, MedGen 320 which provides information related to the human genes, PharmGKB which provides information about influences of the human genetic variation on a drug response, DAILYMED which provides drug information, OpenFDA which summarizes data from the US FDA to provide the data to the public, and CPIC which is provided by the Cancer Prevention Institute of California.

A disease information table 240 is a database table which stores disease related information received from the public database 300.

A drug information table 250 is a database table which stores drug related information received from the public database 300.

A controller 260 reads the digital twin 230, the disease information table 240, and the drug information table 250 to calculate an onset possibility of the susceptible disease and a risk of the drug side effects. The controller 260 is an application program executed in the server computer.

A security checking unit 270 checks the risk of concomitant prescriptions or concurrent medications in advance.

The service unit 400 is an external electronic device which may connect to the digital twin predicting unit 200 via a network. The service unit 400 may be a client computer, a laptop computer, a smart phone, or a tablet PC. In the service unit 400, an external application 410 may be executed or a dedicated application 420 may be executed, and a separate report (for example, a printed material or a PDF file) 430 may be output.

### Mode for Carrying Out the Invention

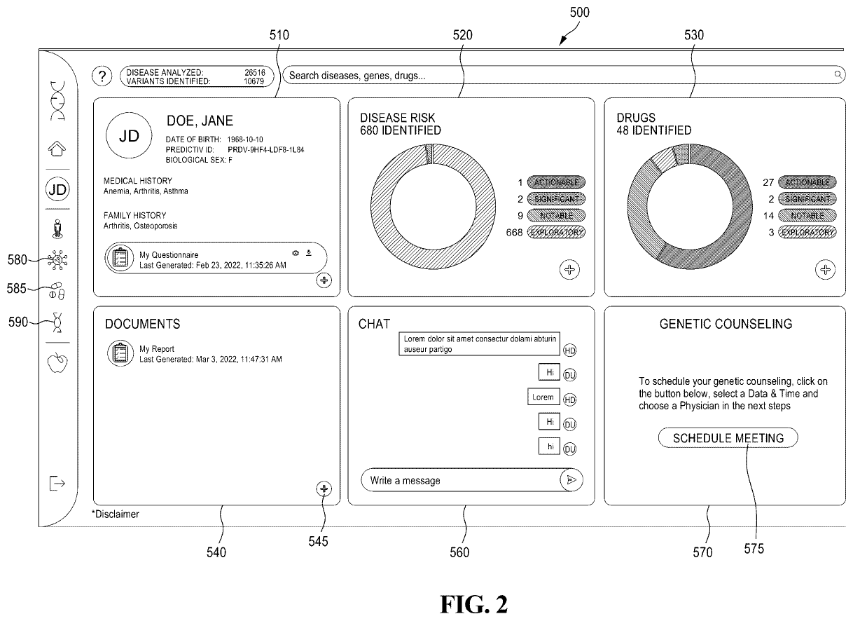

FIG. 2 is a diagram illustrating a main screen 500 of a digital twin system according to an example embodiment of the present disclosure. The main screen 500 is displayed on the service unit 400 of FIG. 1. As illustrated in FIG. 2, the main screen 500 displays a medical record part 510, a disease prediction part 520, a drug side effect prediction part 530, a document part 540, a chatting part 560, and a memo pad 570. On a left menu bar, a susceptible disease icon 580, a drug icon 585, and a genetic information icon 590 are displayed.

In the medical record part 510, personal information of the patient (for example, name, a date of birth, and a personal ID) and physical information (for example, gender, a medical history, and a family history) are displayed.

In the disease prediction part 520, a risk of the patient which catches the susceptible disease is displayed on a graph. For example, 680 diseases are reviewed for the patient. Among them, 668 cases of an exploratory level, 9 cases of a notable level, two cases of a significant level, and one case of actionable level having a high onset possibility are inferred by the predicted candidate group screening AI unit 214 to be displayed.

The drug side effect predicting unit 530 predicts and displays the drug side effects of the patient. For example, 46 drugs are reviewed for the patient. Among them, 3 cases of an exploratory level, 14 cases of a notable level, two cases of a significant level, and 27 cases of actionable level having a high side effect possibility are inferred by the predicted candidate group screening AI unit 214 to be displayed.

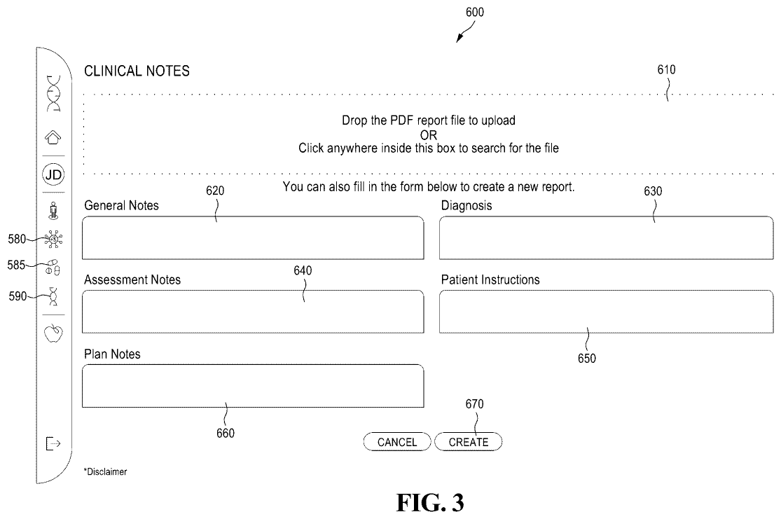

In the document part 540, the document generation button 545 may be clicked to create and input the clinical note 600 as illustrated in FIG. 3.

The chatting unit 560 enables chatting through the message input when the remote medical examination is performed between a patient and a doctor.

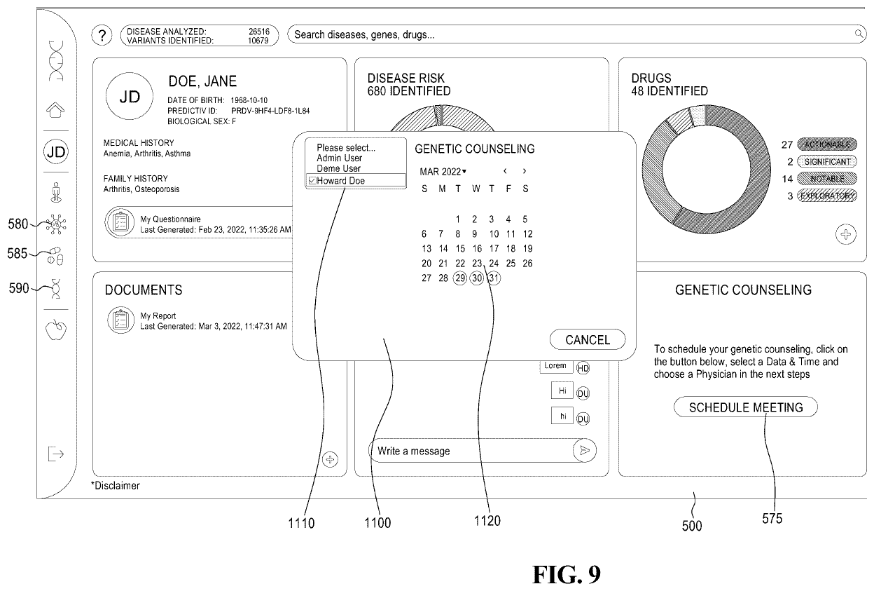

The memo pad 570 enables the selection of a date for a medical appointment as illustrated in FIG. 9 by pressing a scheduling button 575.

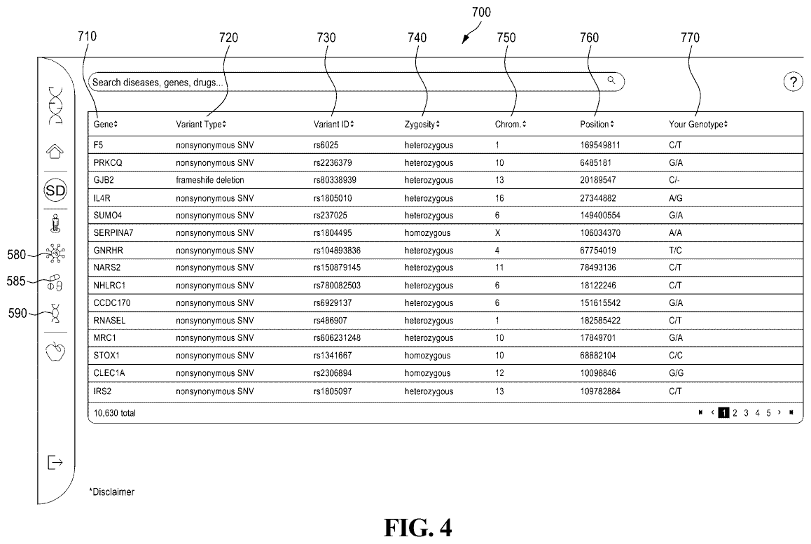

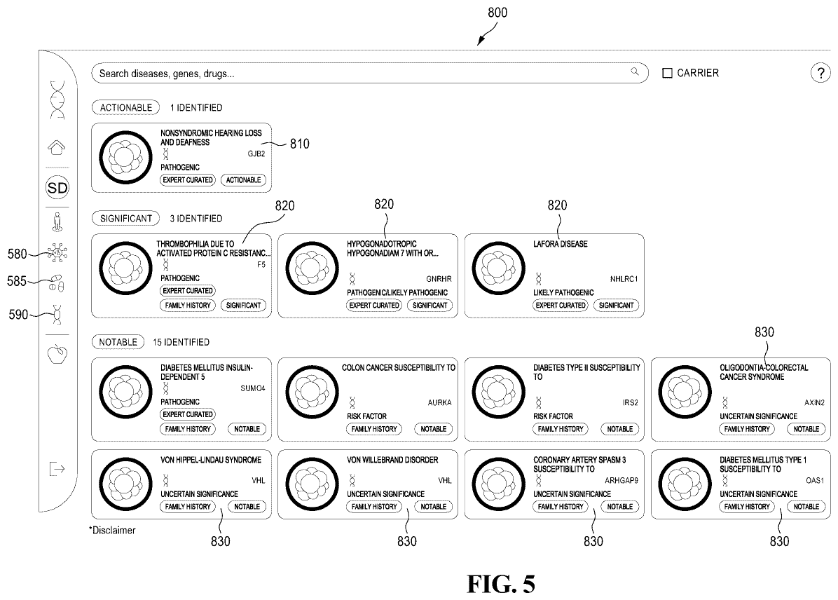

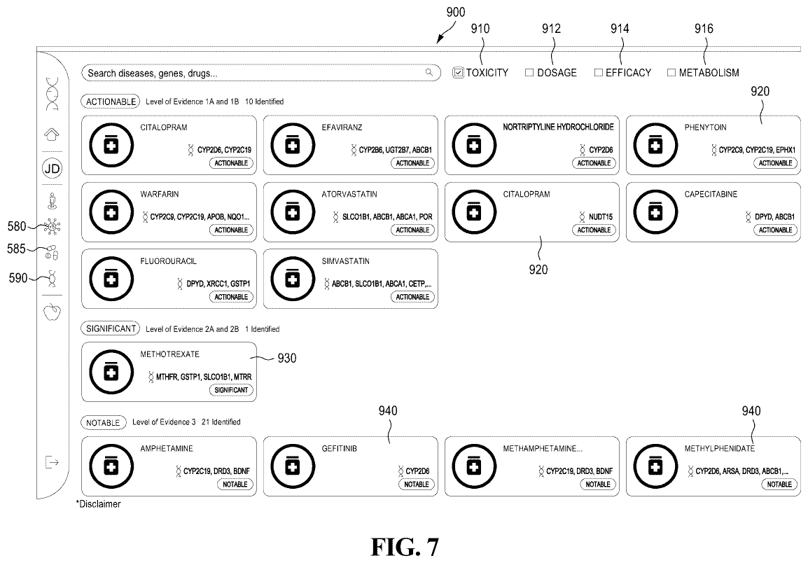

The susceptible disease icon 580 of the left menu bar displays a detailed window screen related to the susceptible disease predicting unit 800 as illustrated in FIG. 5. The drug icon 585 displays a detailed window screen of the drug risk predicting unit 900 as illustrated in FIG. 7. The genetic information icon 590 displays a gene variant list 700 as illustrated in FIG. 4.

FIG. 3 is a diagram illustrating a clinical note unit 600 displayed by a document generation button 545 of FIG. 2 according to an example embodiment. As illustrated in FIG. 3, an uploading unit 610 drags and drops a file to be transmitted to upload the file to a server device of the digital twin predicting unit 200.

A general note unit 620 may write a general memorandum as a text. A diagnosis unit 630 may write a diagnostic record. An assessment unit 640 may input a clinical assessment matter. A patient instruction 650 may input an instruction from the patient. A future plan unit 660 may input a future treatment plan. When a clinical note creation button 670 is pressed, all input information is transmitted and stored to the digital twin predicting unit 200.

FIG. 4 is a diagram illustrating a genetic variant list 700 displayed by a genetic information icon 590 of FIG. 2 according to an example embodiment. As illustrated in FIG. 4, the genetic variant list 700 includes fields of a name of a gene 710, a variant type 720, a variant ID 730, a zygosity type 740, a chromosome number 750, a position 760, and a genotype 770 of a gene.

The name of a gene 710 may be F5, PRKCQ, GJB2, IL4R, and the like, and a total of 10630 genes are provided. The variant type 720 may be nonsynonymous SNV, frameshift deletion, and the like. The variant ID 730 may be rs6025, rs2236379, rs80338939, and the like. The zygosity type 740 may be heterozygous and homozygous, and the like. The chromosome number 750 may be 1, 10, 13, 16, and the like. The gene position 760 may be 169549811, 6485181, 20189547, 27344882, and the like. The genotype 770 may be cytosine C/thymine T, guanine G/adenine A, A/G, A/A, and the like.

FIG. 5 is a diagram illustrating a susceptible disease predicting unit 800 displayed by a susceptible disease icon 580 of FIG. 2 according to an example embodiment. As illustrated in FIG. 5, a first level of susceptible disease 810 is an actionable level having a very high onset possibility and is displayed with red. A second level of susceptible disease 820 is a significant level and is displayed with an orange color. A third level of susceptible disease 830 is a notable level and is displayed with yellow.

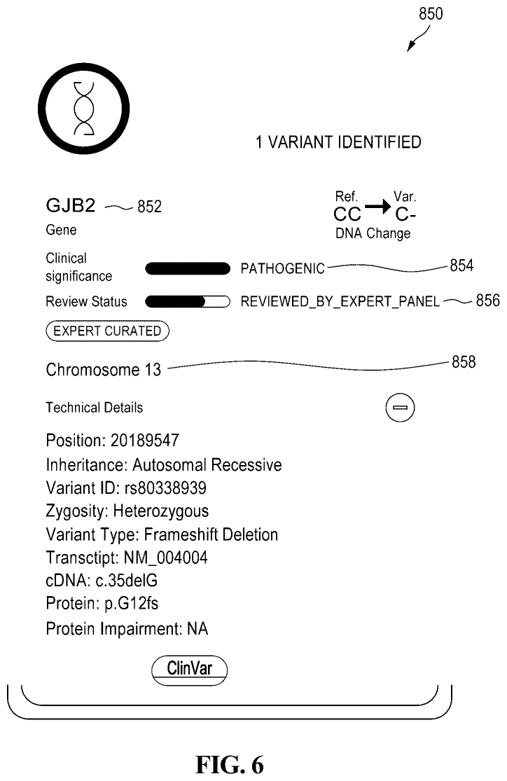

FIG. 6 is a diagram illustrating a detailed disease informatics unit 850 displayed by clicking one disease of FIG. 5 according to an example embodiment. As illustrated in FIG. 6, a name of a variant gene 852 related to the corresponding disease, a clinical significance 854, a review state 856, and a position and detailed information 858 of the gene are displayed.

FIG. 7 is a diagram illustrating a drug risk predicting unit 900 displayed by a drug icon 585 of FIG. 2 according to an example embodiment. As illustrated in FIG. 7, one of a toxicity 910, a dosage 912, an efficacy 914, and a metabolism 916 of the drug may be selected by a checkbox.

When the toxicity 910 is selected, a first level of risk drug 920 is an actionable level having a very high side effect possibility, and is displayed with red. A second level of risk drug 930 is a significant level, and is displayed with an orange color. A third level of risk drug 940 is a notable level, and is displayed with yellow.

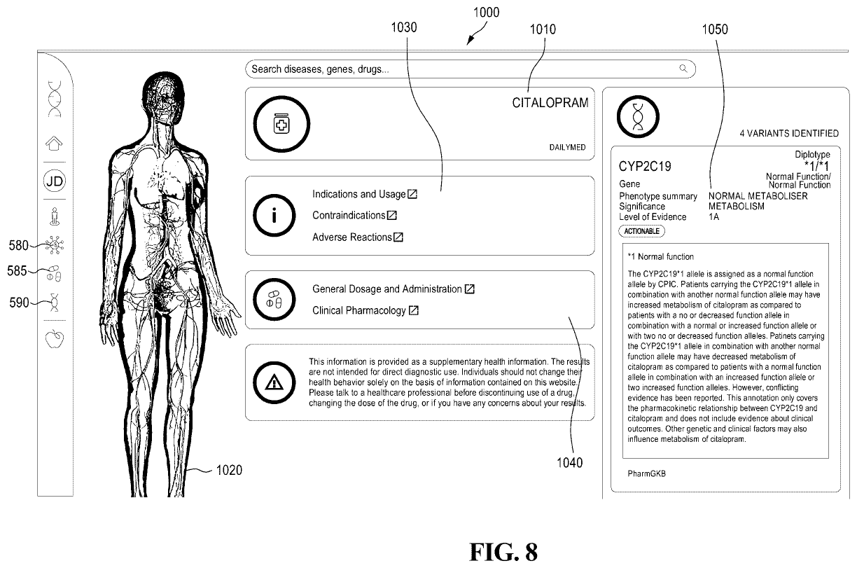

FIG. 8 is a diagram illustrating a detailed drug informatics unit 1000 displayed by clicking one drug of FIG. 7 according to an example embodiment. As illustrated in FIG. 8, a drug name 1010, indication and usage, contraindication, and adverse reaction 1030, a general dosage and administration 1040, and detailed information 1050 are displayed. At one side, a 3D human body avatar 1020 is displayed, and a site for the corresponding drug or the corresponding disease is displayed in blinking. The 3D human body avatar 1020 may be used as a graphic model of the digital twin 230.

FIG. 9 is a diagram illustrating a scheduling window 1100 displayed by clicking a scheduling button 575 of FIG. 2 according to an example embodiment. As illustrated in FIG. 9, after selecting a desired service on the scheduling window 1100 by means of a service selecting unit 1110, a reservation date is selected from a calendar 1120 to schedule a medical appointment.

Optionally, the main screen 500 as illustrated in FIG. 2 may provide a video screen chatting service.

As described above, the detailed description of the exemplary embodiments of the disclosed present disclosure is provided such that those skilled in the art implement and carry out the present disclosure. While the disclosure has been described with reference to the preferred embodiments, it will be understood by those skilled in the art that various changes and modifications of the present disclosure may be made without departing from the spirit and scope of the disclosure. For example, those skilled in the art may use configurations disclosed in the above-described exemplary embodiments by combining them with each other. Therefore, the present disclosure is not intended to be limited to the above-described exemplary embodiments but to assign the widest scope consistent with disclosed principles and novel features.

The present disclosure may be implemented in another specific form within the scope without departing from the spirit and essential feature of the present disclosure. Therefore, the detailed description should not restrictively be analyzed in all aspects and should be exemplarily considered. The scope of the present disclosure should be determined by rational interpretation of the appended claims and all changes are included in the scope of the present disclosure within the equivalent scope of the present disclosure. The present disclosure is not intended to be limited to the above-described exemplary embodiments but to assign the widest scope consistent with disclosed principles and novel features. Further, claims having no clear quoting relation in the claims are combined to configure the embodiment or may be included as new claims by correction after application.

### [Industrial Applicability]->[Description of Main Reference Numerals of Drawings]

100: DNA sequencing analysis unit
110: Sequencing data
200: Digital twin predicting unit
214: Predicted candidate group screening AI unit
216: Bioinformatics pipeline unit
218: Dimensionality reduction AI unit
220: Bioinformatics integration unit
230: Digital twin file
240: Disease information table
250: Drug information table
260: Controller
270: Security checking unit
300: Public database
310: CLINVAR database
320: MedGen database
400: Service unit
410: External application
420: Dedicated application
430: Report

1. A gene-based digital twin system which predicts a medical risk, comprising:
a clinical information generating unit, including:
(i-1) a DNA preprocessing unit which processes genetic sequencing data of a patient in a predetermined format;
(i-2) a bioinformatics pipeline unit which sequentially outputs the preprocessed DNA bio information in a predetermined unit;
(i-3) a predicted candidate group screening AI unit which screens a predicted candidate group for the disease and a predicted candidate group for the drug with respect to the DNA bio information output from the bioinformatics pipeline unit;
(i-4) a dimensionality reduction AI unit which reduces a size of the preprocessed DNA bio information; and
(i-5) a bioinformatics integration unit which receives and integrates the bio information trained by the predicted candidate group screening AI unit and the dimensionality reduction AI unit from the bioinformatics pipeline unit,
(ii) a patient's digital twin file which is virtually generated based on information about a body of the patient, and the predicted candidate group for the disease and the predicted candidate group for the drug received from the bioinformatics integration unit;
(iii) a disease information table which receives and stores information about a disease from the outside;
(iv) a drug information table which receives and stores information about a drug from the outside; and
(v) a controller which predicts and outputs a medical risk of the patient based on the digital twin file, the disease information table, and the drug information table.

2. The gene-based digital twin system which predicts a medical risk of claim 1, wherein the predicted candidate group screening AI unit screens the predicted candidate group for the disease and the predicted candidate group for the drug for a predetermined major gene variant among the DNA bio information.

3. The gene-based digital twin system which predicts a medical risk of claim 2, wherein the predetermined major gene variant includes a list including at least one of a name of the gene, a variant type, a variant ID, a zygosity type, a chromosome number, a position, and a genotype.

4. The gene-based digital twin system which predicts a medical risk of claim 1, wherein the predicted candidate group for the disease is screened by dividing the risk of the disease into a plurality of levels.

5. The gene-based digital twin system which predicts a medical risk of claim 4, wherein the plurality of levels is divided into:
a first level of susceptible disease group having a highest disease onset possibility;
a third level of susceptible disease group having a lowest disease onset possibility; and
a second level of susceptible disease group having a possibility corresponding to an intermediate level between the first level of susceptible disease group and the third level of susceptible disease group.

6. The gene-based digital twin system which predicts a medical risk of claim 1, wherein the predicted candidate group for the drug is screened by dividing the risk of the drug side effects into a plurality of levels.

7. The gene-based digital twin system which predicts a medical risk of claim 6, wherein the plurality of levels is divided into:
a first level of risk drug group having a highest drug side effect possibility;
a third level of risk drug group having a lowest drug side effect possibility; and
a second level of risk drug group having a possibility corresponding to an intermediate level between the first level of risk drug group and the third level of risk drug group.

8. The gene-based digital twin system which predicts a medical risk of claim 1, wherein the predicted candidate group for the drug includes at least one of information about a toxicity, a dosage, an efficacy, and metabolism of the drug.

9. The gene-based digital twin system which predicts a medical risk of claim 1, wherein the digital twin file further includes a 3D human body avatar model.

10. The gene-based digital twin system which predicts a medical risk of claim 9, wherein information about the disease or the drug is displayed on the 3D human body avatar model.
